(19) United States
(12) Patent Application Publication
(10) Pub. No.: US 2023/0181156 A1
(43) Pub. Date: Jun. 15, 2023

(54) AUTOMATIC SEGMENTATION AND TREATMENT PLANNING FOR A VESSEL WITH COREGISTRATION OF PHYSIOLOGY DATA AND EXTRALUMINAL DATA

(71) Applicant: PHILIPS IMAGE GUIDED THERAPY CORPORATION, San Diego, CA (US)

(72) Inventors: Asher COHEN, SAN DIEGO, CA (US); Pei-Yin CHAO, EINDHOVEN (NL); Ronald Christiaan HELMSTRIJD, EINDHOVEN (NL)

(21) Appl. No.: 18/076,459

(22) Filed: Dec. 7, 2022

Related U.S. Application Data
(60) Provisional application No. 63/288,554, filed on Dec. 11, 2021.

(51) Int. Cl. A61B 8/12; A61B 8/08

(52) U.S. Cl. CPC A61B 8/12; A61B 8/0891

(57) ABSTRACT

A system includes a processor circuit that receives physiology measurements obtained by an intraluminal physiology measurement device within a body lumen. The processor circuit automatically segments the body lumen into a plurality of segments based on the physiology measurements. The processor circuit then determines a change in the physiology measurements corresponding to each segment and determines a recommended position for a treatment device within the body lumen based on the change for each segment. The processor circuit provides an output associated with the recommended position on a display.

### CROSS-REFERENCE TO RELATED APPLICATIONS

This application claims priority to and the benefit of U.S. Provisional Application No. 63/288,554, filed Dec. 11, 2021, which is incorporated by reference herein in its entirety.

### TECHNICAL FIELD

The present disclosure relates generally to co-registering data from different medical diagnostic modalities. In particular, physiology data of a vessel, including blood pressure data, may be co-registered to an x-ray-based image and analyzed to automatically segment the vessel and make treatment procedure recommendations.

### BACKGROUND

Physicians use many different medical diagnostic systems and tools to monitor a patient's health and diagnose medical conditions. Different modalities of medical diagnostic systems may provide a physician with different images, models, and/or data relating to internal structures within a patient. These modalities include invasive devices and systems, such as intravascular systems, and non-invasive devices and systems, such as x-ray systems, and computed tomography (CT) systems. Using multiple diagnostic systems to examine a patient's anatomy provides a physician with added insight into the condition of the patient.

In the field of intravascular imaging and physiology measurement, co-registration of data from invasive devices (e.g., intravascular ultrasound (IVUS) devices or instantaneous wave-free ratio (iFR) devices) with images collected non-invasively (e.g., via x-ray angiography) is a powerful technique for improving the efficiency and accuracy of vascular catheterization procedures. Co-registration identifies the locations of intravascular data measurements along a blood vessel by mapping the data to an angiography image of the vessel. A physician may then know exactly where in the vessel a measurement was made, rather than estimate the location.

Currently, a physician tasked with creating a treatment plan manually identifies potential proximal and distal landing zones of treatment devices, such as stents. The physician must determine how many stents to deploy, at which locations, of which diameter, and length. Currently, a user may select regions of a vessel with an input, such as drawing on an input device. The system may then output a change in pressure for the selected region. This method is prone to error. Improper placement of a stent may include placing the stent at the wrong location, placing a stent of the wrong size including a stent that is unnecessarily large, or placing an incorrect number of stents including too many or too few. Any of these mistakes may lead to a failed treatment procedure or complications post deployment.

### SUMMARY

Embodiments of the present disclosure are systems, devices, and methods for automatically segmenting a vessel and automatically recommending the type, size, placement location, and number of stents based on invasive physiology data coregistered to non-invasive x-ray data. The disclosed systems, devices, and methods advantageously assist a physician in several ways. Specifically, they assist a physician in choosing an appropriate stent to remedy restrictions in blood flow, including an appropriate stent length and diameter. They additionally assist a physician in selecting an appropriate number of stents. This advantageously minimizes the amount of stent material deployed within a patient vessel during a treatment procedure, reducing the risk of post-deployment complications. The disclosed systems, devices, and methods also advantageously assist a physician in determining distal and proximal landing zones of the stent. The physician may more accurately ensure that distal and proximal landing zones are placed at healthy regions of the vessel and not placed at locations where side branches branch off from the vessel.

The co-registration system simultaneously receives physiology data, such as pressure data, from within a vessel and x-ray images showing a view of the vessel. The physiology measurement device may also be seen within the x-ray images. Based on the location of the physiology device within each x-ray image, each pressure datum may be associated with the location shown by the position of the physiology device within the x-ray image. The vessel may then be automatically segmented into segments corresponding to high changes in pressure and segments corresponding to little to now change in pressure. A total change in pressure is then determined for each segment. Segments of high changes in pressure which exceed a threshold may be selected as segments of interest. One of the x-ray images may be analyzed to identify locations along the vessel where side branch vessels branch off of the measured vessel using image processing techniques. A processor circuit of the system may receive as inputs the pressure data, the x-ray images, the segments including segments of interest, and the locations of side branches. Based on these inputs, the processor circuit may recommend a number of stents to be deployed to restore blood flow through the entire measured section to a target value. The circuit may also recommend the type of stents, length of stents, and proximal and distal landing zones for each stent. The processor circuit may recommend the locations of proximal and distal landing zones for each stent based on multiple limitations. These limitations may include placing the proximal and distal landing zones in healthy regions of the vessel, or within segments of little to no pressure change. These limitations may include ensuring that proximal and distal landing zones are not placed at the locations of side branches. They may also include ensuring that adjacent stents are placed more than a minimum distance from one another. The limitations may include ensuring that the minimum amount of stent material is placed within the patient vessel.

In an exemplary aspect, a system is provided. The system includes a processor circuit configured for communication with an intraluminal physiology measurement device, wherein the processor circuit is configured to: receive a plurality of physiology measurements obtained by the intraluminal physiology measurement device during a movement of the intraluminal physiology measurement device within a body lumen of a patient, wherein the movement of the intraluminal physiology measurement device defines a region of the body lumen; automatically segment the region based on the plurality of physiology measurements such that the region includes a plurality of segments; determine a change in the physiology measurements corresponding to each segment of the plurality of segments; determine a recommended position within the body lumen for a treatment device based on the change corresponding to a first segment of the plurality of segments; and provide, to a display in communication with the processor circuit, an output associated with the recommended position.

In one aspect, the processor circuit is further configured to compare the change corresponding to each segment of the plurality of segments with a threshold change. In one aspect, the processor circuit is configured to identify first segment in response to the change corresponding to the first segment meeting or exceeding the threshold change. In one aspect, the processor circuit is configured to determine a total change corresponding to all of the plurality of segments. In one aspect, the processor circuit is configured to: compare the total change in the plurality of physiology measurements to a threshold change; determine the recommended position based on the comparison of the total change to the threshold change. In one aspect, the processor circuit is further configured to calculate a predicted total change in the plurality of physiology measurements corresponding to expected physiology measurements to be obtained after the treatment device is positioned within the body lumen and the predicted total change is less than the threshold change. In one aspect, the processor circuit is configured to determine the recommended position such that one or more ends of the treatment device are placed within one or more segments of the plurality of segments corresponding to the change in the plurality of physiology measurements less than a threshold change in the plurality of physiology measurements. In one aspect, the processor circuit is configured to determine an additional recommended position within the body lumen for an additional treatment device, and wherein a distance between the treatment device and the additional treatment device exceeds a threshold distance. In one aspect, the processor circuit is configured to identify a location along the body lumen of a side branch. In one aspect, the processor circuit is configured to determine the recommended position such that the treatment device does not cross a side branch. In one aspect, the output comprises a screen display comprising: a graphical representation of the physiology measurements; a graphical representation of the first segment; and a graphical representation of the treatment device.

In an exemplary aspect, a method is provided. The method includes receiving, with a processor circuit in communication with an intraluminal physiology measurement device, a plurality of physiology measurements obtained by the intraluminal physiology measurement device during a movement of the intraluminal physiology measurement device within a body lumen of a patient, wherein the movement of the intraluminal physiology measurement device defines a region of the body lumen; automatically, with the processor circuit, segmenting the region based on the plurality of physiology measurements such that the region includes a plurality of segments; determining, with the processor circuit, a change in the physiology measurements corresponding to each segment of the plurality of segments; determining, with the processor circuit, a recommended position within the body lumen for a treatment device based on the change corresponding to a first segment of the plurality of segments; and providing, to a display in communication with the processor circuit, an output associated with the recommended position.

In an exemplary aspect, a system is provided. The system includes a processor circuit configured for communication with an intravascular pressure-sensing guidewire, wherein the processor circuit is configured to: receive a plurality of pressure measurements obtained by the intravascular pressure-sensing guidewire during a movement of the intravascular pressure-sensing guidewire within a blood vessel of a patient, wherein the movement of the intravascular pressure-sensing guidewire defines a region of the blood vessel; automatically segment the region based on the pressure measurements such that the region includes a plurality of segments; determine a change in the pressure measurements corresponding to each segment of the plurality of segments; compare the change in pressure measurements for each segment of the plurality of segments to a threshold change in pressure measurements; identify one or more segments of the plurality of segments corresponding to a change in pressure measurements satisfying the threshold change in pressure measurements; determine one or more recommended positions within the blood vessel for one or more stents based on one or more segments of the plurality of segments; and provide, to a display in communication with the processor circuit, an output associated with the one or more recommended positions.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1A is a schematic diagram of a physiology measurement and x-ray system, according to aspects of the present disclosure.

FIG. 1B is a schematic diagram of an extraluminal imaging system, according to aspects of the present disclosure.

FIG. 2 is a schematic diagram of a processor circuit, according to aspects of the present disclosure.

FIG. 3 is a diagrammatic view of a region of a patient vasculature, according to aspects of the present disclosure.

FIG. 4 is a diagrammatic view of a region of a patient vasculature, according to aspects of the present disclosure.

FIG. 5 is a diagrammatic view of a table of data associated with segments of a vessel prior to a treatment procedure, according to aspects of the present disclosure.

FIG. 6 is a diagrammatic view of a region of a patient vasculature, according to aspects of the present disclosure.

FIG. 7 is a diagrammatic view of a table of data associated with segments of a vessel after a treatment procedure, according to aspects of the present disclosure.

FIG. 8 is a diagrammatic view of a graphical user interface, according to aspects of the present disclosure.

FIG. 9 is a flow diagram of a method of automatically segmenting a vessel and generating a treatment plan for the vessel based on coregistration of physiology data and extraluminal data, according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

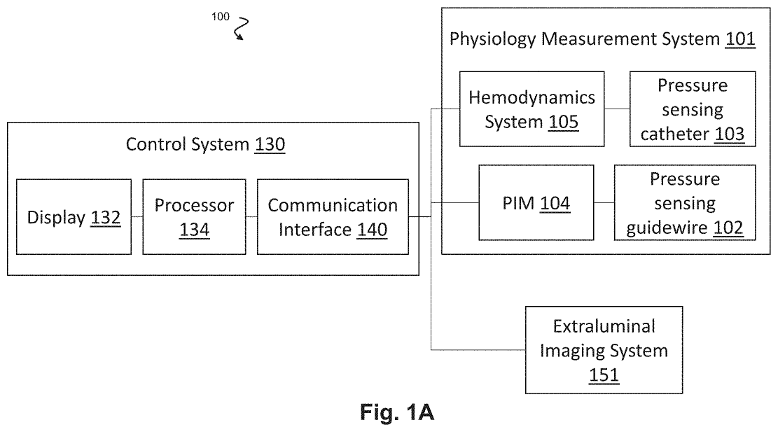

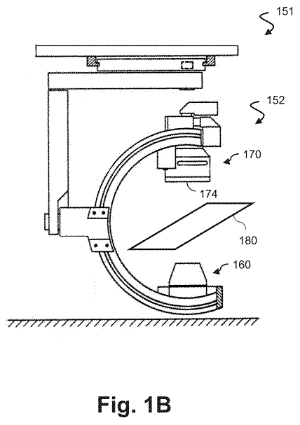

FIG. 1A is a schematic diagram of a physiology measurement and x-ray system 100, according to aspects of the present disclosure. In some embodiments, the physiology measurement and x-ray system 100 may include two separate systems or be a combination of two systems: a physiology measurement system 101 and an extraluminal imaging system 151. The physiology measurement system 101 obtains medical data about a patient's body while an intraluminal device is positioned inside the patient's body. In some embodiments, the intraluminal sensing system 101 may be a physiology measurement system 101. The physiology measurement system 101 can control an intraluminal device to obtain intraluminal data of the inside of the patient's body while the intraluminal device is inside the patient's body. The physiology measurement system 101 may include a hemodynamics system 105, a pressure sensing catheter 103, a patient interface module (PIM) 104, a pressure sensing guidewire 102, and/or other various elements. In some embodiments, the intraluminal device may include the pressure sensing catheter 103 and/or the pressure sensing guidewire 102. The extraluminal imaging system 151 may obtain medical data about the patient's body while the extraluminal imaging device 152 is positioned outside the patient's body. For example, the extraluminal imaging system 151 can control extraluminal imaging device 152 to obtain extraluminal images of the inside of the patient's body while the extraluminal imaging device 152 is outside the patient's body.

The intraluminal system 101 may be in communication with the extraluminal imaging system 151 through any suitable components. Such communication may be established through a wired cable, through a wireless signal, or by any other method of communication. In addition, the intraluminal system 101 may be in continuous communication with the x-ray system 151 or may be in intermittent communication. For example, the two systems may be brought into temporary communication via a wired cable, or brought into communication via a wireless communication, or through any other suitable method at some point before, after, or during an examination. In addition, the intraluminal system 101 may receive data such as x-ray images, annotated x-ray images, metrics calculated with the x-ray imaging system 151, information regarding dates and times of examinations, types and/or severity of patient conditions or diagnoses, patient history or other patient information, or any suitable data or information from the x-ray imaging system 151. The x-ray imaging system 151 may also receive any of these data from the intraluminal imaging system 101. In some embodiments, and as shown in FIG. 1, the intraluminal system 101 and the x-ray imaging system 151 may be in communication with the same control system 130. In this embodiment, both systems may be in communication with the same display 132, processor 134, and communication interface 140 shown as well as in communication with any other components implemented within the control system 130.

In some embodiments, the system 100 may not include a control system 130 in communication with the intraluminal system 101 and the x-ray imaging system 151. Instead, the system 100 may include two separate control systems. For example, one control system may be in communication with or be a part of the intraluminal system 101 and an additional separate control system may be in communication with or be a part of the x-ray imaging system 151. In this embodiment, the separate control systems of both the intraluminal system 101 and the x-ray imaging system 151 may be similar to the control system 130. For example, each control system may include various components or systems such as a communication interface, processor, and/or a display. In this embodiment, the control system of the intraluminal system 101 may perform any or all of the coregistration steps described in the present disclosure. Alternatively, the control system of the x-ray imaging system 151 may perform the coregistration steps described.

The physiology measurement system 101 can be an invasive blood pressure or blood flow measurement system. In some instances, the physiology measurement system 101 can be a pressure ratio system, such as an instant wave-free ratio (iFR) system, a fractional flow reserve (FFR) system, a Pd/Pa system, and/or any other suitable pressure ratio calculation system. The intraluminal system 101 may include a pressure guide wire 102, such as a solid core pressure wire. The pressure wire may include one or more features described in U.S. Pat. No. 5,715,827, granted Feb. 10, 1998, and titled “Ultra Miniature Pressure Sensor and Guide Wire Using the Same and Method,” U.S. Pat. No. 8,277,386, granted Oct. 2, 2012, and titled, “Combination Sensor Guidewire and Methods of Use,” U.S. Pat. No. 9,339,348, granted May 17, 2016, and titled, “Devices, Systems, and Methods for Assessing a Vessel,” all of which are hereby incorporated by reference in their entirety.

At a high level, a pressure sensing device may be positioned within a body lumen of a patient. The pressure sensing device may include a pressure-sensing guidewire 102 and a pressure sensing catheter 103. The pressure-guidewire 102 may include a pressure sensor. The pressure-sensing catheter may also include a pressure sensor. During a pressure pullback procedure, the pressure-sensing catheter 103 may be positioned within the vessel at a location proximal to the region to be measured. The sensor of the pressure-sensing guidewire 102 may also be positioned within the vessel at a position distal to the region to be measured. The pressure-sensing catheter 103 may remain substantially stationary during the pullback procedure. The pressure guidewire 102 is then pulled such that the sensor from the distal position in a proximal direction through the vessel. As the distal guidewire sensor moves through the lumen, both the sensor of the guidewire 102 and the sensor of the catheter 103 collect pressure measurements. Thus, for each position of the guidewire 102, two pressure measurements may be collected: a distal guidewire pressure and a proximal catheter pressure. These two pressures may then be compared to generate a pressure ratio. The pressure ratio may be a fractional flow reserve (FFR), instant wave-free ratio (iFR), Pd/Pa, and/other any other suitable pressure ratio. For example, when the two sensors are substantially in the same place within the vessel (e.g., after a pressure pullback procedure is complete), the pressures recorded by each sensor will be the same or substantially the same. The resulting pressure ratio of these two pressures may therefore by 1.0 or close to 1.0. If the starting location of the pullback is distal of a blockage in the vessel, then the pressure measure by the distal guidewire sensor will be less than the pressure measured by the proximal catheter sensor such that the pressure ratio is less than 1.0. How much less than 1.0 the pressure ratio is provides an indication of the severity of the blockage. As the distal guidewire sensor is moved proximally along the guidewire in the vessel from the starting location (distally within the vessel), the pressure measured by the distal guidewire sensor may to vary with respect to the proximal, stationary catheter sensor. As a result, as the distal guidewire sensor is moved, the ratio may begin to increase such that at different locations along the analyzed vessel and as the distal guidewire pressure sensor approaches the proximal catheter sensor, the pressure ratio corresponding to the location of the distal guidewire pressure sensor approaches 1.0.

The communication interface 140 facilitates communication of measurements between the control system 130 and the physiology measurement system 101. In some embodiments, the communication interface 140 performs preliminary processing of the data prior to relaying the data to the processor 134. In an embodiment, the communication interface 140 also supplies high- and low-voltage DC power to support operation of devices of the physiology measurement system 101.

The PIM 104 may be configured to additionally facilitate communication between the physiology measurement system 101 and the control system 130. For example, the PIM 104 may electrically couple a transmission line bundle to the communication interface 140 and physically couples the any pressure sensing device including the pressure sensor guidewire 102 and/or the pressure sensing catheter 102 to the communication interface 140. In some embodiments, the communication interface 140 may be a PIM.

The hemodynamics system 105 may include various features of the physiology measurement system 101. For example, the hemodynamics system 105 may include a communication interface facilitating communication of the pressures sensing catheter 103 and the control system 130. In some embodiments, the hemodynamics system 105 may be in communication with additional elements of the physiology measurement system 101, such as the pressure sensing guidewire 102, or any other systems or devices. For example, the hemodynamics may be in communication with an extraluminal imaging system, such as the extraluminal imaging system 151. The hemodynamics system 105 can be communication with electrocardiogram (ECG) electrodes and provide a graphical display of an electrocardiogram of the patient's heart. The hemodynamic system 105 can be in communication with a heart rate sensor and provide a graphical display of the heart rate. The hemodynamic system 105 can be in communication with a external blood pressure monitor (e.g., a sphygmomanometer, an inflatable cuff, and/or a manometer) and provide a graphical display of the systolic and diastolic blood pressures.

In some embodiments, the pressure sensing device (e.g., pressure-sensing guidewire 102 and/or pressures sensing catheter 103) obtains intraluminal (e.g., intravascular) pressure data. In some embodiments, the intraluminal system 101 is an intravascular pressure sensing system that determines pressure ratios based on the pressure data, such as fractional flow reserve (FFR), instantaneous wave-free ratio (iFR), and/or other suitable ratios between distal pressure and proximal/aortic pressure (Pd/Pa). In some embodiments, the intraluminal device may be a flow sensing device (e.g., flow-sensing guidewire) that obtains intraluminal (e.g., intravascular) flow data, and the intraluminal system 101 is an intravascular flow sensing system that determines flow-related values based on the pressure data, such as coronary flow reserve (CFR), flow velocity, flow volume, etc.

FIG. 1B is a schematic diagram of an extraluminal imaging system 151, according to aspects of the present disclosure. The x-ray imaging system 151 may include an x-ray imaging apparatus or device 152 configured to perform x-ray imaging, angiography, fluoroscopy, radiography, venography, among other imaging techniques. The x-ray imaging system 151 can generate a single x-ray image (e.g., an angiogram or venogram) or multiple (e.g., two or more) x-ray images (e.g., a video and/or fluoroscopic image stream) based on x-ray image data collected by the x-ray device 152. The x-ray imaging device 152 may be of any suitable type, for example, it may be a stationary x-ray system such as a fixed c-arm x-ray device, a mobile c-arm x-ray device, a straight arm x-ray device, or a u-arm device. The x-ray imaging device 152 may additionally be any suitable mobile device. The x-ray imaging device 152 may also be in communication with the control system 130. In some embodiments, the x-ray system 151 may include a digital radiography device or any other suitable device.

The x-ray device 152 as shown in FIG. 1B includes an x-ray source 160 and an x-ray detector 170 including an input screen 174. The x-ray source 160 and the detector 170 may be mounted at a mutual distance. Positioned between the x-ray source 160 and the x-ray detector 170 may be an anatomy of a patient or object 180. For example, the anatomy of the patient (including the vessel 120) can be positioned between the x-ray source 160 and the x-ray detector 170.

The x-ray source 160 may include an x-ray tube adapted to generate x-rays. Some aspects of the x-ray source 160 may include one or more vacuum tubes including a cathode in connection with a negative lead of a high-voltage power source and an anode in connection with a positive lead of the same power source. The cathode of the x-ray source 160 may additionally include a filament. The filament may be of any suitable type or constructed of any suitable material, including tungsten or rhenium tungsten, and may be positioned within a recessed region of the cathode. One function of the cathode may be to expel electrons from the high voltage power source and focus them into a well-defined beam aimed at the anode. The anode may also be constructed of any suitable material and may be configured to create x-radiation from the emitted electrons of the cathode. In addition, the anode may dissipate heat created in the process of generating x-radiation. The anode may be shaped as a beveled disk and, in some embodiments, may be rotated via an electric motor. The cathode and anode of the x-ray source 160 may be housed in an airtight enclosure, sometimes referred to as an envelope.

In some embodiments, the x-ray source 160 may include a radiation object focus which influences the visibility of an image. The radiation object focus may be selected by a user of the system 100 or by a manufacture of the system 100 based on characteristics such as blurring, visibility, heat-dissipating capacity, or other characteristics. In some embodiments, an operator or user of the system 100 may switch between different provided radiation object foci in a point-of-care setting.

The detector 170 may be configured to acquire x-ray images and may include the input screen 174. The input screen 174 may include one or more intensifying screens configured to absorb x-ray energy and convert the energy to light. The light may in turn expose a film. The input screen 174 may be used to convert x-ray energy to light in embodiments in which the film may be more sensitive to light than x-radiation. Different types of intensifying screens within the image intensifier may be selected depending on the region of a patient to be imaged, requirements for image detail and/or patient exposure, or any other factors. Intensifying screens may be constructed of any suitable materials, including barium lead sulfate, barium strontium sulfate, barium fluorochloride, yttrium oxysulfide, or any other suitable material. The input screen 374 may be a fluorescent screen or a film positioned directly adjacent to a fluorescent screen. In some embodiments, the input screen 174 may also include a protective screen to shield circuitry or components within the detector 170 from the surrounding environment. In some embodiments, the x-ray detector 170 may include a flat panel detector (FPD). The detector 170 may be an indirect conversion FPD or a direct conversion FPD. The detector 170 may also include charge-coupled devices (CCDs). The x-ray detector 370 may additionally be referred to as an x-ray sensor.

The object 180 may be any suitable object to be imaged. In an exemplary embodiment, the object may be the anatomy of a patient. More specifically, the anatomy to be imaged may include chest, abdomen, the pelvic region, neck, legs, head, feet, a region with cardiac vasculature, or a region containing the peripheral vasculature of a patient and may include various anatomical structures such as, but not limited to, organs, tissue, blood vessels and blood, gases, or any other anatomical structures or objects. In other embodiments, the object may be or include man-made structures.

In some embodiments, the x-ray imaging system 151 may be configured to obtain x-ray images without contrast. In some embodiments, the x-ray imaging system 151 may be configured to obtain x-ray images with contrast (e.g., angiogram or venogram). In such embodiments, a contrast agent or x-ray dye may be introduced to a patient's anatomy before imaging. The contrast agent may also be referred to as a radiocontrast agent, contrast material, contrast dye, or contrast media. The contrast dye may be of any suitable material, chemical, or compound and may be a liquid, powder, paste, tablet, or of any other suitable form. For example, the contrast dye may be iodine-based compounds, barium sulfate compounds, gadolinium-based compounds, or any other suitable compounds. The contrast agent may be used to enhance the visibility of internal fluids or structures within a patient's anatomy. The contrast agent may absorb external x-rays, resulting in decreased exposure on the x-ray detector 170.

In some embodiments, the extraluminal imaging system 151 could be any suitable extraluminal imaging device, such as computed tomography (CT) or magnetic resonance imaging (MRI).

When the control system 130 is in communication with the x-ray system 151, the communication interface 140 facilitates communication of signals between the control system 130 and the x-ray device 152. This communication includes providing control commands to the x-ray source 160 and/or the x-ray detector 170 of the x-ray device 152 and receiving data from the x-ray device 152. In some embodiments, the communication interface 140 performs preliminary processing of the x-ray data prior to relaying the data to the processor 134. In examples of such embodiments, the communication interface 140 may perform amplification, filtering, and/or aggregating of the data. In an embodiment, the communication interface 140 also supplies high- and low-voltage DC power to support operation of the device 152 including circuitry within the device.

The processor 134 receives the x-ray data from the x-ray device 152 by way of the communication interface 140 and processes the data to reconstruct an image of the anatomy being imaged. The processor 134 outputs image data such that an image is displayed on the display 132. In an embodiment in which the contrast agent is introduced to the anatomy of a patient and a venogram is to be generated, the particular areas of interest to be imaged may be one or more blood vessels or other section or part of the human vasculature. The contrast agent may identify fluid filled structures, both natural and/or man-made, such as arteries or veins of a patient's vascular system, including cardiac vasculature, peripheral vasculature, neural vasculature, renal vasculature, and/or any other suitable lumen inside the body. For example, the x-ray device 152 may be used to examine any number of anatomical locations and tissue types, including without limitation all the organs, fluids, or other structures or parts of an anatomy previously mentioned. In addition to natural structures, the x-ray device 152 may be used to examine man-made structures such as any of the previously mentioned structures.

The processor 134 may be configured to receive an x-ray image that was stored by the x-ray imaging device 152 during a clinical procedure. The images may be further enhanced by other information such as patient history, patient record, IVUS imaging, pre-operative ultrasound imaging, pre-operative CT, or any other suitable data.

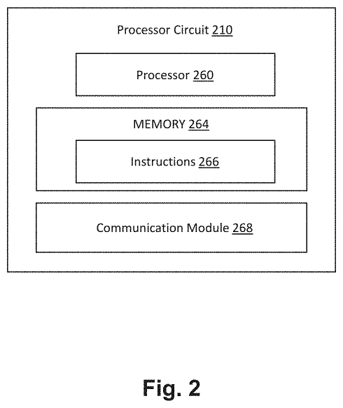

FIG. 2 is a schematic diagram of a processor circuit, according to aspects of the present disclosure. The processor circuit 210 may be implemented in the control system 130 of FIG. 1A, the physiology measurement system 101, and/or the x-ray imaging system 151, or any other suitable location. In an example, the processor circuit 210 may be in communication with pressure sensing device (e.g., the pressure sensing guidewire 102 and/or the pressure sensing catheter 103), the x-ray imaging device 152, the display 132 within the system 100. The processor circuit 210 may include the processor 134 and/or the communication interface 140 (FIG. 1A). One or more processor circuits 210 are configured to execute the operations described herein. As shown, the processor circuit 210 may include a processor 260, a memory 264, and a communication module 268. These elements may be in direct or indirect communication with each other, for example via one or more buses.

The processor 260 may include a CPU, a GPU, a DSP, an application-specific integrated circuit (ASIC), a controller, an FPGA, another hardware device, a firmware device, or any combination thereof configured to perform the operations described herein. The processor 260 may also be implemented as a combination of computing devices, e.g., a combination of a DSP and a microprocessor, a plurality of microprocessors, one or more microprocessors in conjunction with a DSP core, or any other such configuration.

The memory 264 may include a cache memory (e.g., a cache memory of the processor 260), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 264 includes a non-transitory computer-readable medium. The memory 264 may store instructions 266. The instructions 266 may include instructions that, when executed by the processor 260, cause the processor 260 to perform the operations described herein with reference to the probe 110 and/or the host 130 (FIG. 1A). Instructions 266 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 268 can include any electronic circuitry and/or logic circuitry to facilitate direct or indirect communication of data between the processor circuit 210, the probe 110, and/or the display 132 and/or display 132. In that regard, the communication module 268 can be an input/output (I/O) device. In some instances, the communication module 268 facilitates direct or indirect communication between various elements of the processor circuit 210 and/or the probe 110 (FIG. 1A) and/or the host 130 (FIG. 1A).

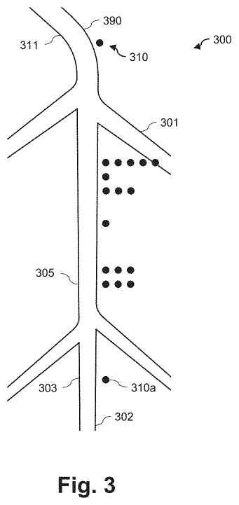

FIG. 3 is a diagrammatic view of a region 300 of a patient vasculature, according to aspects of the present disclosure. FIG. 3 shows a body lumen 390 under measurement. The lumen 390 in FIG. 3 may be any type of lumen. It may be a blood vessel in the human body such as a coronary vessel, a peripheral vessel, or any other lumen, including anatomical and/or man-made structures. In some embodiments, as shown in FIG. 3, the vessel 390 imaged may have multiple side branches 301. A side branch 301 may include a body lumen, such as a vessel. In some embodiments, a side branch 301 may be any type of lumen, as described with reference to the vessel 390. In some embodiments, a side branch 301 may include a body lumen physically coupled to the vessel 390. In some embodiments, a side branch 301 may be a region of the vessel 390.

One aspect of the present disclosure may include acquiring physiological data of the lumen 390. For example, the physiological data can be iFR data. This iFR data may be coregistered to an angiogram roadmap. Various aspects of coregistering physiological data to an extraluminal image may include one or more features described in U.S. Patent Publication No. 2006/0241465, filed Jan. 11, 2006, and titled “Vascular Image Co-registration” which is hereby incorporated by reference in its entirety. Physiology and x-ray co-registration is used to determine locations of pressure measurements in a coronary artery or other body lumens. Accurate physiology and x-ray co-registration may be based on wire tracking.

At a high level, an angiogram image may include a view of the vessel 390. More specifically, the angiogram image may include a view of the region 300. In some embodiments, the angiogram image may be referred to as an angiogram roadmap. The angiogram roadmap image may be one of the extraluminal images acquired by the system 151 of FIG. 1. The angiogram roadmap image may be acquired during a pressure pullback procedure or may be acquired a different time. The roadmap image may depict the vessel to be measured. In an example in which iFR data is coregistered to an extraluminal view of the vessel, an iFR pullback may be performed within a particular vessel while extraluminal images are obtained depicting the vessel and the iFR device. The iFR device may include various radiopaque markers which may be visible within the extraluminal images acquired by the system 151. As a particular pressure measurement is made, that measurement may be associated with the location at which the iFR device is observed by the system 151 at that time. As a result, each pressure measurement received during a pressure pullback may be associated with at least one location within the angiogram roadmap image. In addition, each location along the vessel measured may be associated with one or more pressure measurements. The locations associated with all the pressure measurements made during a pressure pullback procedure may define a pathway. The pathway may extend along the vessel measured. In some embodiments, this pathway, with its corresponding pressure measurements may be overlaid over an extraluminal image. In some embodiments, the pathway may align with a vessel within the extraluminal image. In some embodiments, the extraluminal image may be an extraluminal image obtained with contrast such that vessels of the patient are visible within the image. In some embodiments, this extraluminal image may be the roadmap image previously described or may be a separate image. In some embodiments, the pathway may align with the shape of a guidewire within an extraluminal image. In some embodiments, the extraluminal image may be obtained without contrast. As a result of this coregistration as described, the system may display intraluminal data, such as pressure data overlaid on an extraluminal image at the locations at which the data was collected. In an example in which iFR data is coregistered to an extraluminal images, a resulting display for a user may contain an extraluminal image depicting the region of the vessel measured. The system may display iFR data, such as a pressure ratio or percentage, in response to a user selecting any location along the measured region of the vessel.

Various methods may be used for displaying pressure measurements to a user of the system. For example, one method may include displaying one or more graphical elements 310. The graphical elements 310 shown in FIG. 3 may be of any suitable appearance and shape. In some embodiments, the graphical elements 310 may be dots, although they are only displayed as such for pedagogical purposes. The graphical elements 310 may be positioned adjacent to the vessel 390 measured and may correspond to a change in pressure ratio. In some embodiments, the graphical elements 310 may be positioned near locations along the vessel 390. In this way, the position of the graphical elements 310 may indicate to a user locations within the vessel 390 at which a pressure change is measured.

In one example as shown in FIG. 3, a distal location 302 may be the distal most position measured by the iFR device. At this location, an iFR measurement associated with the location 302 may be some measurement less than 1.0. For example, a sensor of the pressure sensing guidewire (e.g., the pressure sensing guidewire 102) may be positioned at the location 302. A sensor of the pressure sensing catheter 103 may be positioned at a proximal location (e.g., a proximal location 311). A difference between the pressure measured at the location 311 and the location 302 may be displayed as a ratio of some value less than 1.0. However, as the distal sensor of the pressure sensing guidewire 102 is moved in a proximal direction from the location 302 toward the location 311, the ratio may change at, for example, the location 303. At the location 303 shown, the pressure ratio may increase by, for example, 0.01. This 0.01 change in the pressure ratio may be illustrated by the dot 310a shown placed next to the location 303. The graphical elements 310 may each correspond to a 0.01 change in the pressure ratio or may refer to any other amount of change. As shown, the pressure ratio may remain the same from the location 303 to the location 305 at which point, as shown by the additional graphical elements 310 at this location, the ratio may again increase. Assuming each graphical element 310 represents a 0.01 change, the new pressure ratio as measured at the location 305 may increase by 0.03, increased by 0.01 at the location 303 as shown by the dot 304 and increased by 0.03 at the location 305 as shown by the three graphical elements 310 at that location. The other graphical elements 310 shown in FIG. 3 may illustrate similar changes in the pressure ratio.

In some embodiments, a view of the region 300 including one or more of the graphical elements 310 may be displayed to a user. A depiction of the vessel 390 with accompanying graphical elements 310 may be included in a view, such as a graphical user interface, displayed to a user on the display 132 (FIG. 1A).

Other methods of displaying pressure data may include displaying one or more pressure ratios. For example, pressure data, including pressure ratio values may be displayed adjacent to a measured vessel overlaid over an extraluminal image included within a graphical user interface. In some embodiments, the processor circuit (e.g., the processor circuit 210) may be configured to display one or more pressure data ratios at multiple locations along the vessel. For example, the processor circuit 210 may be configured to display one, two, three, four, or more pressure ratio values simultaneously. In some embodiments, the processor circuit 210 may be configured to automatically display any of these pressure ratio values adjacent to the measured vessel overlaid over an extraluminal image when the extraluminal image is displayed to a user. In some embodiments, the processor circuit 210 may be configured to display one or more pressure ratio values in response to a user input. For example, a user may select a location along the measured vessel within the extraluminal image. The processor circuit 210 may be configured to identify a selected location and identify a pressure ratio obtained during a pressure pullback procedure and stored in a memory (e.g., the memory 264) corresponding to the selected location. The pressure ratio value may be displayed within the extraluminal image adjacent to, or near, the selected location. In some embodiments, the user may select multiple locations and the processor circuit 210 may display pressure ratio values associated with all the selected locations simultaneously.

In some embodiments, pressure data may be displayed in other ways. For example, pressure data may be displayed as percentages. In such an embodiment, a pressure sensor measurement of a distal sensor of a pressure sensing guidewire 102 (FIG. 1A) that is equal to a pressure sensor measurement of a proximal sensor of a pressure sensing catheter 103 (FIG. 1A) may be displayed as a pressure percentage value of 100%. Differences in pressure measurements between these two sensors may be determined as percentages less than 100%. These percentage values may be displayed near their corresponding locations along a measured vessel according to any suitable method, including any of those methods described with reference to pressure ratio values. As an example, pressure percentage values may be displayed automatically or in response to a user input.

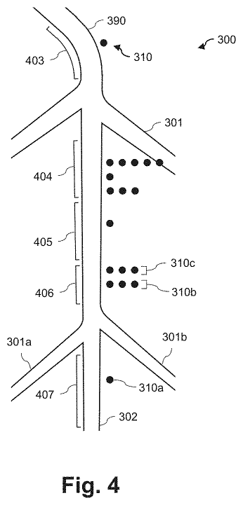

FIG. 4 is a diagrammatic view of a region 300 of a patient vasculature, according to aspects of the present disclosure. FIG. 4 shows the same region 300 of the vessel segmented according to pressure data. The processor circuit 210 may receive as inputs the pressure measurement data collected by the iFR device during a pullback procedure, any extraluminal images from the extraluminal imaging system 151, a roadmap image, data relating to the presence and location of side branches 301, and data corresponding to the coregistration of the pressure data to the angiogram roadmap or any other extraluminal image.

The processor circuit 210 may obtain information relating to side branch location in any way. For example, the circuit 210 may receive the location of side branches as a user input. For example, the user may select on an input device, such as a touch screen, by clicking with a mouse or other pointer device, or with any other device, locations of side branches 301 along the measured vessel 390 as shown in the roadmap. In some embodiments, the processor circuit 210 may automatically determine the locations of side branches 301 by any suitable method. For example, the processor 210 may use various image processing techniques, such as edge detection, to determine the locations of side branches 301. The processor circuit 210 may be configured to use any number of image processing techniques to identify features of an extraluminal image and/or a vessel, such as the vessel 390. The processor circuit 210 may be configured to use image processing techniques such as edge identification of radiopaque markers of intravascular devices, pixel-by-pixel analysis to determine transition between light pixels and dark pixels, image editing or restoration, filtering such as linear filtering or other filtering methods, image padding, or any other suitable techniques. In some embodiments, the processor circuit may use various artificial intelligence methods including deep learning techniques such as neural networks or any other suitable techniques to identify the locations of side branches 301 within an image.

Using any of these inputs, the processor circuit 210 may automatically segment the vessel as shown in the roadmap. The circuit may segment the vessel based on pressure measurements, the locations of side branches, or other parameters. As one example shown in FIG. 4, the processor circuit 210 may identify a segment 407. This segment 407 may have been determined by the processor circuit 210 to be a region without any side branches 301 and is representative of no significant change in pressure. For example, as shown by the presence of only one graphical element 310 (specifically, graphical element 310a corresponding to a 0.01 increase in pressure ratio values along the segment 407), the processor circuit may determine that this region is to be designated as a segment 407.

In some embodiments, the processor circuit 210, or a user, may designate a threshold amount of change in pressure ratio at which a segment will be created. For example, the system may compare changes in pressure ratio values to a threshold of 0.02. For example, if for a given region of the vessel 390, the pressure ratio changes by less than 0.02, that region of the vessel may be included in one segment. Such may be the case shown by the segment 407 in FIG. 4. As shown by the single graphical element 310a in FIG. 4 along segment 407, the region of vessel 390 shown by the segment 407 may be identified by the processor circuit 210 as one segment.

In some embodiments, the processor circuit 210 may identify separate segments at side branches, as shown. In other words, any locations at which a side branch 301 separates from the measured portion of the vessel 390 may not be included in any of the segments of the vessel 390. For example, a location at which a side branch 301 separates from the measured region of a vessel 390 may define an end point of a segment. As shown in FIG. 4 for example, a proximal end of the segment 407 may be defined by the processor circuit 210 by the location at which the side branch 301a meets the measured region of the vessel 390. Similarly, the segment 406 may include as a distal end point a location proximate to the side branch 301a and the side branch 301b between the segment 406 and the segment 407. Shown proximal to segment 406 is segment 405 and segment 404. Because segment 405 represents a region of little to no pressure ratio change between segments 404 and 406, segment 405 may be designated as its own segment. In contrast, segments 404 and 406 relate to areas of the vessel corresponding to greater changes and are designated by the processor circuit 210 as separate segments from the segment 405.

In some embodiments, a user of the system may determine various parameters of automatic segmentation, such as the value of the threshold change in pressure relating to a single segment, a minimum distance along the vessel for a segment, a maximum distance along the vessel for a segment, or other parameters. In this way, if a user observes, based on the extraluminal images received and/or coregistered pressure data, that more or less segments are needed, he or she may alter the minimum distance, maximum distance, and/or threshold change in pressure allowing for additional or reduced segmentation respectively. For example, with an adjustment to various parameters, the segment 404 may be split into multiple segments. Alternatively, segments 404, 405, and/or 406 may be combined according to various combinations to suit the preference or needs of the user.

As an example, the segment 406 is described. As shown in FIG. 4 and as shown by the presence of more than one graphical element 310 associated with the segment 406, the regions of the vessel 390 as shown by the segment 406 may be grouped or assigned to the same segment 406. In some embodiments, the region of the vessel 390 associated with the segment 406 may represent a distance along the vessel 390 that exceeds a minimum distance threshold. For example, if no minimum distance threshold is determined, or if the minimum distance threshold is very low, at regions of the measured vessel 390 corresponding to greater changes in pressure ratio, more segments than are practical may be created.

For example, at the region 406, six graphical elements 310 are shown, including a set of three graphical elements 310b at a distal location and a set of three graphical elements 310c at a proximal location. At the distal location corresponding to the graphical elements 310b, the processor circuit 210 may determine that the threshold change in pressure ratio values has been met. For example, if a threshold pressure ratio change is 0.01, a change of 0.03 may be represented by the elements 310b. The processor circuit 210 may then designate the region from the intersection of the side branches 301a and 301b with the measured portion of the vessel to the distal location of the elements 310b as one segment. Similarly, as shown by the three elements 310c, the processor circuit may designate this proximate location as a separate segment. However, in some embodiments, a user of the system, or the processor circuit 210, may determine that two separate segments corresponding to the elements 310b and 310c are too many or make the display or analysis of the pressure data unwieldy or impractical. A minimum distance parameter may, therefore, be adjusted to ensure that the elements 310b and 310c are included in the same segment. For example, a distance threshold may be determined and/or received by the processor circuit 210. In the example previously described, a segment corresponding to only the graphical elements 310b (e.g., extending from the side branches 301a/301b to the elements 310b) may not exceed the minimum distance threshold. However, the distance from the side branches 301a/301b to the elements 310c may exceed the threshold. As a result, this region, including the elements 310b and 310c may be included within a single segment, segment 406.

Similarly, a maximum distance threshold may be determined to define a proximal endpoint of the segment 406. A maximum distance threshold may limit the length of a given segment. In some embodiments, distance thresholds, such as the minimum and maximum distance thresholds described may be applied only after a change in pressure ratio threshold is exceeded. In such an embodiment, regions of the measured vessel which do not exceed a threshold pressure ratio change may be of any suitable length while regions of the vessel which exceed the threshold pressure ratio change may be limited by the minimum and maximum segment distance thresholds.

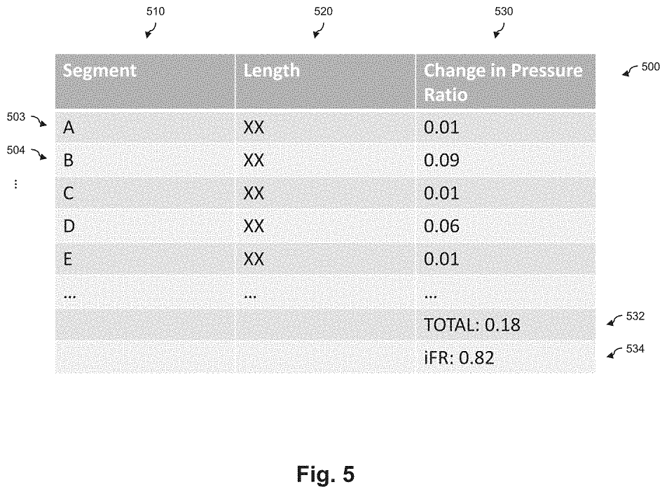

These parameters may be similarly applied to all regions of the measured vessel 390 to define the segments 405, 404, and 403, as well as any other segments. As described with reference to FIG. 5, data associated with the regions of the vessel corresponding to these various segments may be analyzed to determine optimal treatment procedure types and/or locations.

FIG. 5 is a diagrammatic view of a table 500 of data associated with segments of a vessel prior to a treatment procedure, according to aspects of the present disclosure. The table 500 shown in FIG. 5 includes a column 510 identifying a segment, a column 520 identifying a length measurement associated with each segment, and a column 530 identifying a total pressure ratio change measurement corresponding to the segments listed in column 510.

The column 510 may include one or more labels associated with each of the segments described with reference to FIG. 4. Referring again to FIG. 4, in some embodiments, the processor circuit 210 (FIG. 2) may be configured to assign each segment (e.g., segment 403, segment 404, etc.) a unique label. As an example, and as shown in FIG. 5, the segment 403 may be assigned a label “A,” the segment 404 may be assigned a label “B,” the segment 405 may be assigned a label “C,” the segment 406 may be assigned a label “D,” and the segment 407 may be assigned a label “E.” It is noted that additional labels may be created. For example, additional segments distal of the segment 407 of FIG. 4 may be identified by the processor circuit. These additional distal segments may be assigned labels in a similar way. For example, an additional segment adjacent to the segment 407 may be assigned a label “F” and so on. It is also noted that the labels assigned to the segments shown in FIG. 4 may be of any suitable type. For example, they may include any suitable characters, numbers, variables, or any alpha-numeric text and may be assigned in any particular order. For example, the segments may be assigned labels in an alphabetical and/or numerical order from a most proximal segment to a most distal segment or vice versa.

Still referring to FIG. 4, in some embodiments, the processor circuit 210 may be additionally configured to display the assigned labels and any associated markers with the segments within the view of the vessel 390 for a user. For example, the processor circuit 210 may be configured to display an extraluminal image with or without contrast to the user. This image may include markers identifying the locations of any of the segments o FIG. 4. These markers may include visual identifiers such as those shown in FIG. 4. A marker identifying a segment may identify a distal end point of a segment and a proximal end point of a segment. They may include any visual elements or characteristics including symbols such as arrows, lines, dots, or any other visual elements. The processor circuit 210 may be additionally configured to display the label associated with each segment in conjunction with or simultaneously with the markers identifying the locations of the segments. These labels may be the same labels shown in FIG. 5 in the column 510.

The column 520 may indicate a length associated with each segment. The processor circuit 210 may determine a length measurement for each identified segment along the vessel. In some embodiments, the processor circuit 210 may identify a length of each segment using coregistration information or other information from an extraluminal image. For example, the processor circuit 210 may be configured to identify one or more radiopaque portions of an intravascular device within an extraluminal image. For example, a pressure sensing guidewire (e.g., the guidewire 102) and/or a pressure sensing catheter (e.g., the catheter 103) may include radiopaque markers visible within an extraluminal image acquired while either of these devices are positioned within the body lumen (e.g., the vessel 390). The distance between these radiopaque markers may be known. The processor circuit 210 may be configured to determine the number of pixels between these radiopaque markers. In this way, a pixel within an extraluminal image may be associated with a distance measurement and the processor circuit 210 may determine a distance measurement of a length based on the number of pixels associated with the particular length within the image. The processor circuit 210 may determine the length of each segment shown in FIG. 4 and store these length measurements in a memory in conjunction with their respective segments as shown in the table 500.

The processor circuit 210 may also determine a total change in pressure ratio through each segment of FIG. 4. This data may be displayed in the column 530 of the table 500 in a row corresponding to each segment. As an example, the change in pressure ratio for segment A (e.g., the segment 403 of FIG. 4) may be displayed in a row 503 of the table 500. The change in pressure ratio for segment B (e.g., the segment 404 of FIG. 4) may be displayed in a row 504 of the table 500, and so on. The change in pressure ratio for segment A may be 0.01, as shown by the presence of one graphical element 310 along the segment 403 in FIG. 4. Similarly, the total change in pressure ratio for segment B may be 0.09, as shown by the presence of nine graphical elements 310 along the segment 404 in FIG. 4. Similar total changes in pressure ratios may be displayed in respective rows associated with their appropriate segments in the column 530. In some implementations, the change in pressure ratio for a given segment may be a difference between the pressure ratio measured at one end of the segment (e.g., a distal end) and the pressure ratio measure at the other end of the segment (e.g., a proximal end).

In some embodiments, the processor circuit 210 may also be configured to determine a total change in pressure ratio for the entire region of the vessel measured. In the example shown in FIG. 4 and FIG. 5, this total change in pressure ratio is 0.18 as shown by the value 532 in table 500 of FIG. 5. Subtracting this amount from the maximum pressure ratio of 1.0, yields a total iFR of 0.82 for the region of the vessel measured, as shown by the value 534.

In some embodiments, a physician may determine a target total iFR. This target may be determined by the user of the system 100 or may be based on recommendations of experts in the field. In some embodiments, a target total iFR may be provided and/or displayed by the processor circuit 210 based on recommendations. In some examples, the target total iFR may be 0.89. In some examples, the target total iFR may be 0.95. The target total iFR may be any other value. In some embodiments, the processor circuit 210 may recommend various treatment methods and/or devices, as well as locations for treatment methods or devices within the measured vessel based on a comparison of the current total iFR 534 shown in FIG. 5 and any target total iFR.

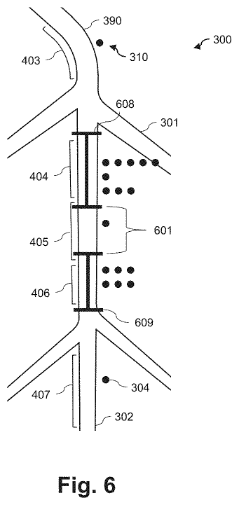

FIG. 6 is a diagrammatic view of a region 300 of a patient vasculature, according to aspects of the present disclosure. The view shown in FIG. 6 may include depictions of virtual stents. Depictions of virtual stents may be displayed to a user of the system 100 before a treatment procedure. In some embodiments, the processor circuit 210 may overlay depictions of virtual stents over an extraluminal image in location corresponding to recommended locations of stents. In this way, the processor circuit 210 may be configured to automatically identify and display locations of stents. The processor circuit 210 may additionally be configured to automatically recommend a type of stent as well as a stent diameter, stent length, a distal landing zone of a stent, a proximal landing zone of a stent, and/or a location of a stent along a vessel.

In the example shown in FIG. 6, a stent 608 and a stent 609 may be positioned along the vessel 390. The recommendation of stents may be performed by the processor circuit 210 after the circuit 210 has automatically segmented the vessel 390.

As shown in FIG. 6, the processor circuit may recommend to a user to place one or more stents along one or more segments of the vessel to restore blood flow. The recommendation of a stent may be based on any number of suitable inputs or desired outcomes. For example, desired outcomes of stent recommendation may include positioning the proximal and distal stent ends (e.g., proximal and distal landing zones) in healthy tissue, ensuring that the proximal and distal stent ends are not positioned at locations of the vessel 390 where side branches 301 separate from the measured portion of the vessel, and minimizing the amount of stent material placed in a patient's anatomy. In some cases, greater amounts of stent material (e.g., metal) placed within a patient lumen may correspond to an increased risk of complications of the stent post deployment. To mitigate the risk of post-deployment complications, as little stent material as possible is desired to achieve optimal results. To achieve this goal of minimizing stent material, the system may recommend stents of the least length possible while sufficiently remedying any constrictions. An optimal length of stents may be determined to restore blood flow by determining the minimum amount of stent to insert into the vessel while predicting a non-ischemic result. Another desired outcome may be to place adjacent stents apart from one another by a specified minimum distance (e.g., 5 mm). Another desired outcome may be to achieve a target iFR, such as 0.95 or any other target number. To ensure that two or more of the desired outcomes listed herein are met, the processor circuit 210 may receive, as an input, pressure data received from the pressure-sensing device and/or extraluminal image data. The processor circuit may determine a recommended position of a stent based on pressure data such that a central portion of a stent is placed at an area where changes in pressure ratio are observed. The processor may also ensure that distal and proximal ends of the stent are placed in healthy regions of the vessel, or regions exhibiting little to no change in pressure ratio measurements. The processor may also recommend a stent placement based on extraluminal image data in that a stent is placed so as not to overlap a side branch 301 of the vessel.

When determining a stent placement recommendation, the processor circuit 210 may identify various segments of interest. These segments of interest may correspond to segments of the vessel associated with large changes in pressure ratio. In some embodiments, a threshold change in pressure ratio may be determined. Segments of the vessel which exceed this threshold may be identified by the processor circuit 210 as segments of interest. For example, if a threshold change in pressure ratio is set to be 0.03, segments 404 and 406 (FIG. 4), also referred to as segments B and D in FIG. 5, may be selected by the processor circuit 210 as segments of interest, while segments 403 (segment A in FIG. 5), segment 405 (segment C in FIG. 5), and segment 407 (segment E in FIG. 5) may not be selected as segments of interest. The processor circuit 210 may be configured to recommend a placement of one or more stents along any identified segments of interest.

In some embodiments, a difference between the total current iFR (e.g., value 534 in FIG. 5) and the target iFR (e.g., 0.95 or another target iFR value) may be determined. If the current total iFR 534 (FIG. 5) is less than the target iFR, the processor circuit 210 may select segments as segments of interest beginning with the segment corresponding to the highest change in pressure ratio until the combined changes in pressure ratio of the selected segments meets or exceeds the difference between the current iFR and the target iFR. These selected segments may be identified as segments of interest. In the example shown in FIG. 5, for example, the difference between the target iFR and the current iFR 534, with a target iFR of 0.95, is 0.13. As a result, segment 404 (FIG. 4), also referred to as segment B in FIG. 5, may be initially selected as a segment of interest. However, because the change in pressure ratio of segment 404/segment B does not meet or exceed the difference of 0.13, an additional segment, such as segment 406/segment D, may be selected. Combined, the pressure ratio change for segment 404/segment B and segment 406/segment D is 0.15, which exceeds the difference of 0.13 between the current iFR 534 and the target iFR. Segments of interest may also be identified in any other suitable way.

In some embodiments, the selected segments of interest may be displayed to a user in a way which accentuates or highlights the selected segments. For example, by using a bracket or other symbol, varying colors, shading, highlighting, or transparencies to distinguish them. These visual characteristics may be applied to the roadmap angiogram image, to the textual or numeric characters or measurements or in any other way. In some embodiments, the processor circuit 210 may receive as inputs: (1) angiogram images created using conventional x-ray, (2) pressure data recorded using software algorithms or invasive pressure wires, (3) pressure pullback gradients along the length of a vessel, (4) pressure cut-off values to indicate healthy physiology, (5) Optimal stent length and location, and/or (6) other inputs.

In some embodiments, as shown in FIG. 6, depictions of stents may be displayed to a user. For example, as shown in FIG. 6, a visual representation of recommended stents may be displayed. For example, a stent 608 is shown placed over the vessel 390 at segment 404. This visual representation may be accompanied by any suitable data. For example, the processor circuit 210 may recommend to a user the length of the stent 608, the type of stent, the diameter of the stent, and the locations along the vessel 390 for distal and proximal landing zones. In some embodiments, the distal and proximal landing zones may correspond to the distal and proximal ends of the segment 404. In other embodiments, the distal and proximal landing zones may extend past either side of the segment 404. For example, a recommended distal landing zone of stent 608 may be placed within the segment 405, the segment 405 corresponding to a healthy region of the vessel which does not overlap with a side branch 301. In some embodiments, a segment may be determined to correspond to a healthy region of the vessel if the total change in pressure ratio for the segment is below a threshold value. A proximal landing zone of the stent 608 may be placed proximal of the proximal end of the segment 404 according to a similar method. A similar stent 609 is shown corresponding to the segment 406. The length, type, diameter, and locations of distal and proximal landing zones of the stent 609 may be determined in a similar way as described with reference to the stent 608.

In some embodiments, a distance 601 between the distal landing zone of stent 608 and the proximal landing zone of stent 609 may be determined. This distance measurement may be calculated by the processor circuit 210 by any suitable way, including those described previously. The processor circuit 210 may be configured to recommend positions of the stents 608 and 609 such that the distance 601 does not exceed a minimum threshold. In some embodiments, the minimum threshold distance between adjacent stent may be 5 mm. In some embodiments, this minimum threshold distance may be any other value. In some embodiments, the distance 601 may alternatively correspond to the edges of segments as opposed to landing zones of stents. In other embodiments, the distance 601 may correspond to the final distal and/or proximal locations of stents after expansion. Physiologic guided stenting, such as the recommendations shown and describe with reference to FIG. 6, has shown to drive better outcomes for patients. Aspects of the present disclosure assist a physician by automatically identifying segments of interest based on co-registered physiological pullback data in order to optimize stent placement and length during PCI Procedures.

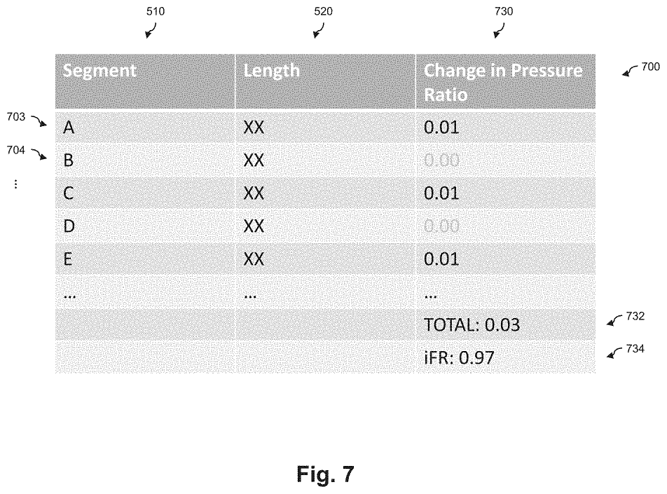

FIG. 7 is a diagrammatic view of a table 700 of data associated with segments of a vessel after a treatment procedure, according to aspects of the present disclosure. The table 700 of FIG. 7 includes the columns 510 and 520 of table 500 described with reference to FIG. 5. The columns 510 and 520 may correspond to segment labels and segment length measurements respectively. Column 730 included in the table 700 may include predicted pressure ratio change measurements for each segment after one or more treatment devices (e.g., stents) are deployed during a treatment procedure. The processor circuit 210 may determine a predicted change in pressure ratio for each segment after deployment of the recommended stents. These predicted change in pressure ratio measurements may be based on virtual stent placement, as shown in FIG. 6. In some embodiments, actual pressure ratio measurements may be performed after the placement of one or more stents in the vessel. In such embodiments, the table 700 may include actual pressure ratio change measurements for each segment. These actual measurements may be displayed simultaneously with the predicted change in pressure ratio measurements of column 730. These measurements may be compared with pre-stent measurements.

As an example, as shown in FIG. 7, the predicted change in pressure ratio of segment 404 (FIG. 4), referred to as segment B in FIG. 7, and segment 406 (FIG. 4), referred to as segment D in FIG. 7, after the placement of stents may be 0.00. Based on these new predicted measurements, a new total predicted change in pressure ratio value 732 may be determined to be 0.03 corresponding to a total iFR value 734 of 0.97. This value would exceed the target total iFR value of 0.95. The circuit may, therefore, confirm that the recommended stent locations, lengths, and types successfully restore blood flow in this manner. The tables 500 and 700 of FIGS. 5 and/or 7 respectively may or may not be displayed to a user. However, the data shown in the tables 500 and 700 may be stored in a memory in communication with the processor circuit 210.

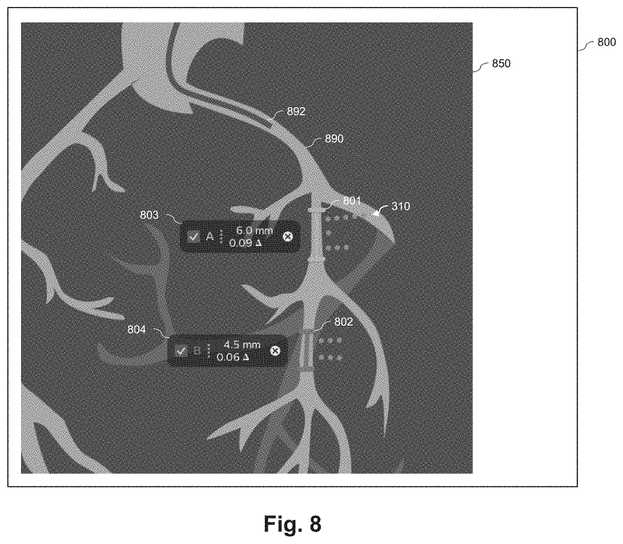

FIG. 8 is a diagrammatic view of a graphical user interface 800, according to aspects of the present disclosure. The graphical user interface 800 may include an image 850 which may be displayed to a user. The image 850 may be an angiogram image or other extraluminal image obtained by the extraluminal imaging system 151. The image 850 may be obtained with or without contrast. In some embodiments, as shown in FIG. 8, the image 850 may be a stylized version of the measured vessel or a stylized version of an extraluminal image. The stylized version may be a simplified or drawn version of an angiogram image. The stylized image may be based on various measurements of an extraluminal image, pressure measurements of the vessel, intravascular ultrasound images, or other measurements or data.

As shown in the graphical user interface 800, the image 850 may include a depiction of a vessel 890. In some embodiments, the vessel 890 may be a vessel which was measured by an intraluminal device, such as a pressure sensing device. In some embodiments, the vessel 890 may be the vessel 390 described with reference to FIG. 3. In some aspects, the image 850 may also include a depiction of a measurement or treatment device 892.

In some embodiments, the image 850 may include one or more visual elements 310. The visual elements 310 may be displayed and positioned adjacent to the measured vessel 890 at locations corresponding to a change in the pressure ratio. The visual elements 310 may be positioned over the image 850 according to aspects of placement of the visual elements 310 described with reference to FIG. 3. In some embodiments, the visual elements 310 are placed by the vessel in the interface 800 only if they correspond to a selected segment of interest. In other embodiments, all visual elements 310 may be shown. The interface 800 may additionally display one or more depictions of virtual stents. For example, a virtual stent 801 and a virtual stent 802 are shown. Adjacent to these virtual stents, the processor circuit may display data corresponding to the region covered by the virtual stent. For example, data 803 may correspond to the stent 801 and data 804 may correspond to the stent 802. Data 803 and/or 804 may include any suitable data including a length measurement of the corresponding stent, a change in pressure ratio along the length of the stent, or additional data. Additional data may include the type of stent recommended, locations of distal or proximal landing zones, a diameter of the stent, a label for the stent (e.g., A or B as shown in FIG. 8 and/or FIG. 5 or FIG. 7, or any other label) and various selectable features. The stents 801 and 802 may be visually differentiated in any way, such as by color, pattern, or any of the other ways described herein.

In some embodiments, data, such as data 803 and 804 may be displayed corresponding to segments as opposed to virtual stents. For example, any of the segments A, B, C, D, and/or E shown and described with reference to FIG. 5 and FIG. 7 may displayed overlaid over the image 850. Similar data including any of the data of included in the data 803 and/or 804 may be displayed in conjunction with any of these segments. As described herein, the processer circuit 210 may output optimal stent position and length data and display these optimal recommendations directly on an angiographic roadmap (e.g., the image 850) creating a PCI plan. The automated outputs may be visible directly on the angiographic roadmap. Operators may interpret the automated outputs visually to guide a stenting strategy.

The processor circuit 210 can determine the segments and/or the proximal landing zone and/or the distal landing zone for the stents 801 and 802 based on the intravascular pressure data and/or the extraluminal imaging data. In some embodiments, the location of the proximal landing zone and/or distal landing zone of the stent 801 and/or the stent 802 can be based on a local maximum change in the pressure ratio. The local maximum change in the pressure ratio can be locations where the pressure ratio increases or decreases quickly (e.g., a large change in pressure ratio over a short distance). For example, the proximal landing zone and/or the distal landing zone can be a threshold distance spaced away from the locations of the maximum change in the pressure ratio, to ensure that the proximal and distal stent ends are within healthy tissue.

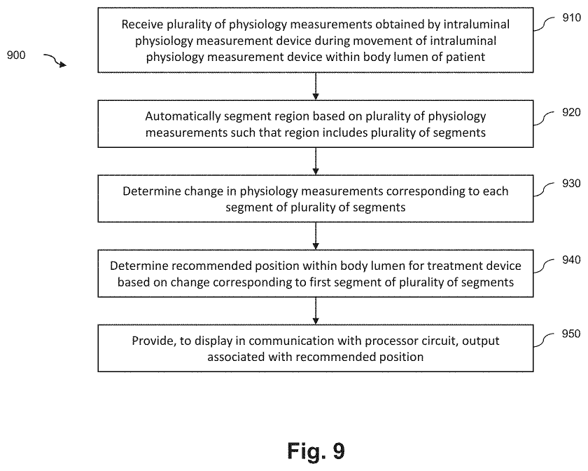

FIG. 9 is a flow diagram of a method 900 of automatically segmenting a vessel and generating a treatment plan for the vessel based on coregistration of physiology data and extraluminal data, according to aspects of the present disclosure. The method 900 may describe an automatic segmentation of a vessel to detect segments of interest using co-registration of invasive physiology and x-ray images. As illustrated, the method 900 includes a number of enumerated steps, but embodiments of the method 900 may include additional steps before, after, or in between the enumerated steps. In some embodiments, one or more of the enumerated steps may be omitted, performed in a different order, or performed concurrently. The steps of the method 900 can be carried out by any suitable component within the diagnostic system 100 and all steps need not be carried out by the same component. In some embodiments, one or more steps of the method 900 can be performed by, or at the direction of, a processor circuit of the diagnostic system 100, including, e.g., the processor 260 (FIG. 2) or any other component.

At step 910, the method 900 includes receiving a plurality of physiology measurements obtained by the intraluminal physiology measurement device during a movement of the intraluminal physiology measurement device within a body lumen of a patient. The movement of the intraluminal physiology measurement device defines a region of the body lumen. In some examples, the step 910 may include receiving a plurality of pressure measurements obtained by the intravascular pressure-sensing guidewire during a movement of the intravascular pressure-sensing guidewire within a blood vessel of a patient and the movement of the intravascular pressure-sensing guidewire may define a region of the blood vessel.

At step 920, the method 900 includes automatically segmenting the region based on the plurality of physiology measurements such that the region includes a plurality of segments. In some examples, the step 920 may include automatically segmenting the region based on pressure measurements such that the region includes a plurality of segments.

At step 930, the method 900 includes determining a change in the physiology measurements corresponding to each segment of the plurality of segments. In some examples, the step 930 may include determining a change in pressure measurements corresponding to each segment of the plurality of segments.

At step 940, the method 900 includes determining a recommended position within the body lumen for a treatment device based on the change corresponding to a first segment of the plurality of segments. In some examples, the step 940 may include comparing the change in pressure measurements for each segment of the plurality of segments to a threshold change in pressure measurements. The processor circuit may also identify one or more segments of the plurality of segments corresponding to a change in pressure measurements satisfying the threshold change in pressure measurements and determine one or more recommended positions within the blood vessel for one or more stents based on one or more segments of the plurality of segments.

At step 950, the method 900 includes providing, to a display in communication with the processor circuit, an output associated with the recommended position. In some examples, the method 900 may include providing an output associated with one or more recommended positions.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A system, comprising:
a processor circuit configured for communication with an intraluminal physiology measurement device, wherein the processor circuit is configured to:
receive a plurality of physiology measurements obtained by the intraluminal physiology measurement device during a movement of the intraluminal physiology measurement device within a body lumen of a patient, wherein the movement of the intraluminal physiology measurement device defines a region of the body lumen;
automatically segment the region based on the plurality of physiology measurements such that the region includes a plurality of segments;
determine a change in the physiology measurements corresponding to each segment of the plurality of segments;
determine a recommended position within the body lumen for a treatment device based on the change corresponding to a first segment of the plurality of segments; and
provide, to a display in communication with the processor circuit, an output associated with the recommended position.

2. The system of claim 1, wherein the processor circuit is further configured to compare the change corresponding to each segment of the plurality of segments with a threshold change.

3. The system of claim 2, wherein the processor circuit is configured to identify first segment in response to the change corresponding to the first segment meeting or exceeding the threshold change.

4. The system of claim 1, wherein the processor circuit is configured to determine a total change corresponding to all of the plurality of segments.

5. The system of claim 4, wherein the processor circuit is configured to:
compare the total change in the plurality of physiology measurements to a threshold change;
determine the recommended position based on the comparison of the total change to the threshold change.

6. The system of claim 5,
wherein the processor circuit is further configured to calculate a predicted total change in the plurality of physiology measurements corresponding to expected physiology measurements to be obtained after the treatment device is positioned within the body lumen,
wherein the predicted total change is less than the threshold change.

7. The system of claim 1, wherein the processor circuit is configured to determine the recommended position such that one or more ends of the treatment device are placed within one or more segments of the plurality of segments corresponding to the change in the plurality of physiology measurements less than a threshold change in the plurality of physiology measurements.

8. The system of claim 1, wherein the processor circuit is configured to determine an additional recommended position within the body lumen for an additional treatment device, and wherein a distance between the treatment device and the additional treatment device exceeds a threshold distance.

9. The system of claim 1, wherein the processor circuit is configured to identify a location along the body lumen of a side branch.

10. The system of claim 9, wherein the processor circuit is configured to determine the recommended position such that the treatment device does not cross a side branch.

11. The system of claim 1, wherein the output comprises a screen display comprising:
a graphical representation of the physiology measurements;
a graphical representation of the first segment; and
a graphical representation of the treatment device.

12. The system of claim 1, wherein the plurality of physiology measurements comprises a plurality of pressure measurements.

13. A method, comprising:
receiving, with a processor circuit in communication with an intraluminal physiology measurement device, a plurality of physiology measurements obtained by the intraluminal physiology measurement device during a movement of the intraluminal physiology measurement device within a body lumen of a patient, wherein the movement of the intraluminal physiology measurement device defines a region of the body lumen;
automatically segmenting, with the processor circuit, the region based on the plurality of physiology measurements such that the region includes a plurality of segments;
determining, with the processor circuit, a change in the physiology measurements corresponding to each segment of the plurality of segments;
determining, with the processor circuit, a recommended position within the body lumen for a treatment device based on the change corresponding to a first segment of the plurality of segments; and
providing, to a display in communication with the processor circuit, an output associated with the recommended position.

14. A system, comprising:
an intravascular pressure-sensing guidewire; and
a processor circuit configured for communication with the intravascular pressure-sensing guidewire, wherein the processor circuit is configured to:
receive a plurality of pressure measurements obtained by the intravascular pressure-sensing guidewire during a movement of the intravascular pressure-sensing guidewire within a blood vessel of a patient, wherein the movement of the intravascular pressure-sensing guidewire defines a region of the blood vessel;
automatically segment the region based on the pressure measurements such that the region includes a plurality of segments;
determine a change in the pressure measurements corresponding to each segment of the plurality of segments;
compare the change in pressure measurements for each segment of the plurality of segments to a threshold change in pressure measurements;
identify one or more segments of the plurality of segments corresponding to a change in pressure measurements satisfying the threshold change in pressure measurements;
determine one or more recommended positions within the blood vessel for one or more stents based on one or more segments of the plurality of segments; and
provide, to a display in communication with the processor circuit, an output associated with the one or more recommended positions.
